(19) United States
(12) Patent Application Publication
(10) Pub. No.: US 2017/0128495 A1
(43) Pub. Date: May 11, 2017

(54) Method of Correcting Pathological Human Skin Conditions Related to Aging

(71) Applicants: OSVILT CORPORATION LIMITED, Nicosia (CY); OOO "Proryvnye Innovatsionnye Tekhonologii", Moscow (RU)

(72) Inventors: Anna N. SOKOLOVA, Yaroslavl (RU); Olga ERSHOV, Netanya (IL)

(21) Appl. No.: 15/412,467

(22) Filed: Jan. 23, 2017

Related U.S. Application Data
(63) Continuation of application No. PCT/RU2014/001000, filed on Dec. 26, 2014.

(30) Foreign Application Priority Data
Jul. 21, 2014 (RU) 2014129926

(51) Int. Cl. A61K 35/33; A61B 10/02; A61B 5/00; C12Q 1/68; C12N 5/077

(52) U.S. Cl. CPC A61K 35/33; C12Q 1/6876; C12N 5/0656; A61B 5/441; A61B 10/02; C12Q 2600/158; C12N 2510/00; C12Q 2600/156

(57) ABSTRACT

A method correcting pathological human skin conditions caused by aging provides reduction of clinical signs of skin aging and improves functional skin parameters by using the patient's autogenous fibroblasts and their further introduction to the patient, where material is sampled, and cells are grown with further isolation of the patient's fibroblast culture. Genetic research of the fibroblast culture is conducted by determining the DNA sequence and the activity of the genes selected from the group including TGFB1, TGFBR2, COL1A1, COL1A2, SOD1, SOD2, GPX1, GPX3, CLCA2. Findings are compared with normal DNA sequences and the data of the normal expression level of the respective genes. Genetic constructs are created with the cDNAs of the patient's genes the activity of which is modified, or the DNA structure of which has deviations. These genetic constructs are embedded in the patient's fibroblast culture. Then these modified autogenous fibroblasts are injected to the patient.

### RELATED APPLICATIONS

This Application is a Continuation application of International Application PCT/RU2014/001000, filed on Dec. 26, 2014, which in turn claims priority to Russian Patent Applications No. RU2014129926, filed Jul. 21, 2014, both of which are incorporated herein by reference in their entirety.

### FIELD OF THE INVENTION

The invention relates to medicine and may be used to treat and rejuvenate the human skin.

### BACKGROUND OF THE INVENTION

Skin aging is a biological process including both structural and functional changes. The structural changes include reduction of collagen quantity, thinning of the subcutaneous fat layer, whereas functional changes include elasticity loss and melanogenesis stimulation.

There are several causes of these processes:

accumulation of toxic metabolites, a higher level of free radical formation, oxidative damage (Dermatoendocrinology 2012, 4(3), 227-231). One of the main causes is the accumulation of mutations in the genome—both point and chromosome ones. (Tokai J Exp Clin Med 2010, 35(4) 152-164; Exp Dermatol 2001, 10(4) 272-279; Exp Dermatol 2004, 13(11), 691-699). In general, the accumulation of somatic mutations—both point mutations, deletions and translocations of chromosome regions—is one of the causes of the body aging in whole (Mutat. Res. 1995, 338 (1-6), 25-34, Trends Genet. 2008, 24(2) 77-85).

There are different approaches to the correction of age-related skin changes.

There methods for correcting age-related skin changes (Facial Plastic Surgery 1999, 1(3), 165-170; Dermatol Surg 2007, 33(3) 263-268; Cell Transpl 2008, 17(7) 775-783; Aesthetic Medicine Bulletin 2011, 10(2), 16-26; RU Patent No 2382077) that involve introduction to the treatment area of the suspension of fibroblasts, which, as a rule, are autogenous in order not to cause the immune response from the patient's body. The basic principle of these methods is to introduce to the body the population of fibroblasts capable to perform their specialized function of synthesising collagen and glycosaminoglycans and to strengthen it due to the cell quantity replenishment. Fibroblasts are injected together with the culture fluid containing different enhancing factors, which promotes the process of proliferation of the patient's own fibroblasts, as well as the synthesis of structural skin components.

This method is efficient only in case the patient's fibroblasts have no genetic defects, and the loss of fibroblasts or their activity reduction are caused only by external factors.

We also know the decision on RU Patent No 2320720<<Method for fibroblast cultivation for replacement therapy>> including the isolation of cells and their incubation in the culture medium. Thereat, fibroblasts are isolated from human skin biopsy samples by their enzymatic treatment in the solution DMEM/5% FBS (Fetal Bovine Serum)/0.2% of dispase/0.1 mg/ml of type I collagenase with the constant stirring and pipetting at the temperature of 37° C. during 1.5 h in sterile conditions, and the obtained cell suspension is centrifuged for 5 min at 200 g, the supernatant is drained, the precipitated out cells are re-suspended in the medium of DMEM/10% FBS/100 U/ml of penicillin, 100 U/ml of streptomycin, 100 U/ml of fungizone and plated, then fibroblasts are set for long cultivation in the medium with the patient's own blood serum:DMEM/10% POBS (Patient's Own Blood Serum)/100 U/ml of streptomycin, 100 U/ml of fungizone or in the therapeutic medium AIM-V. Before the introduction to the patients, the cells are rinsed several times in Krebs-Ring buffer, treated with the solution containing 0.25% of trypsin/0.02% EDTA, to remove the cells from the substrate 1 ml of Krebs-Ringer is added, and then centrifuged for 5 min at 200 g, the supernatant is drained, the cells are re-suspended in a new proportion of Krebs-Ringer solution to be injected to the patients. The cultivated fibroblasts are additionally treated with the cultivation medium with added rhTGF-β 1 in the concentration of 5 ng/ml in order to increase the quantity of myofibroblasts. The obtained suspension is introduced by injection.

However, this method, like the one described above, is efficient only in case the fibroblasts have no genetic defects, and the loss of fibroblasts or their activity reduction are caused only by external factors.

We also know the decision on WO Patent 2004048557 A, which, for the purpose of the therapy of skin defects, including those caused by aging, proposes to introduce the suspension of autologous fibroblasts containing biological agents that may activate fibroblasts. A similar methodology is described in U.S. Pat. No. 5,660,850.

We also know the decision on RU Patent No 2373941<<The method for correcting age-related and pathological changes of human skin covering>>. The method under this patent includes the use of autogenous fibroblasts with the addition of hyaluronic acid; thereat, autogenous fibroblasts in a physiological solution are injected in the area of skin treatment or rejuvenation, and the skin covering is preliminarily treated with the growth medium of fibroblasts with biologically active agents—growth medium factors, including among others fibroblast growth factors. For that purpose, 0.5 ml of suspension containing 1 mln fibroblasts in a physiological solution is prepared with the addition of 0.5 ml of a hyaluronic acid solution in the concentration of 0.02-0.1%; the suspension is stirred and syringed in the amount of 0.1-0.2 ml in the area of treatment or rejuvenation. The gel includes either the serum-free growth medium that contained fibroblasts during 14-28 hours, with the content of BAS 49%, hyaluronic acid or sodium hyaluronate 0.5-1.5%, glycerine 49.548.5%, aromatising agent 0-0.5%, or the growth medium that contained fibroblasts during 14-28 days with the serum, with the content of BAS 49%, hyaluronic acid or sodium hyaluronate 0.5-1.5%, glycerine 49.5-48.5%, aromatising agent 0-0.5%. Before the consumption, the growth medium factors are concentrated on the basis of well-known methods or lyophilized. The growth medium factors are added to the gel in the volume of 0.5-3%, the gel also consists of 70% of glycerine, 1.5% of cattle collagen, 1.5% of hyaluronic acid, and the required quantity of purified water up to 100%.

However, this method, like the one described above, is efficient only in case the fibroblasts have no genetic defects, and the loss of fibroblasts or their activity reduction are caused only by external factors.

In case the deficiency of structural skin components and/or functional changes are caused by the damaged gene expression or mutations in the gene structure changing the activity or function of the protein coded by this gene, this decision is not very efficient as it is not sufficient to simply replenish the cell quantity, but it is also necessary to compensate for their defect. Genetic defects in the prototype were not researched, the fibroblasts used were autogenous ones with the use of hyaluronic acid.

### SUMMARY OF THE INVENTION

The object of the invention is the creation of a method for correcting pathological human skin conditions caused by aging, which will take account of genetic defects, as well as damaged gene expressions, if any, or mutations in the gene structure changing the activity or function of proteins coded by these genes.

The solution aims not simply at the cell quantity replenishment, but also at the compensation for their genetic defects.

The problem may be solved due to the fact that the method for correcting pathological human skin conditions caused by aging, which includes the use of the patient's own fibroblasts and their further introduction to the patient, involves prior assessment of the patient's skin covering, then the material sampling and growing of cells with the further isolation of the patient's fibroblast culture, then the separation of the obtained cell material, the first part of the fibroblast culture being sent for research, and the second part being stored. Then the analysis of the fibroblast culture is made by determining the DNA sequence and the gene activity, followed by the comparison of the findings with the normal group data and the discovery of deviations in the fibroblast genome. On the basis of the genetic research findings the conclusion is made on the connection of the discovered deviations in the gene structure and/or function with the patient's skin changes; thereafter actions are taken to compensate for these deviations, which specifically involves the creation of genetic constructs with cDNA of the patient's genes the activity of which is changed, or the DNA structure of which has deviations, ensuring the structure and functions of the said genes are as those of normal ones. These genetic constructs are embedded in the patient's fibroblast culture, then the autogenous fibroblasts modified with the genetic constructs containing the cDNA of the patient's genes the activity of which is changed, or the DNA structure of which has deviations are injected to the patient. The created genetic constructs with the cDNA of the patient's genes the activity of which is changed, or the DNA structure of which has deviations, are transfected into the patient's cell culture with the help of the viral or nonviral vector or using other well-known not described method, which is apparent for a specialist of any level. The skin covering is assessed using the functional diagnostics with the application of measuring instruments that allow obtaining the quantitative parameters characterising the patient's skin condition. Material is sampled in the area protected from ultraviolet. During the comparison of the findings with normal group data to reveal a deviation in the fibroblast genome, the norm is taken as the gene sequences given in the GenBank database, as well as the gene expression data in the UniGene database.

### Embodiment of the Invention

The invention is embodied by means of the following actions:

1. Running the functional diagnostics of the patient's skin using the measuring instruments that allow obtaining quantitative parameters characterising the patient's skin condition.

2. Sampling the biopsy material in the area protected from ultraviolet, for example, behind the ear.

3. Growing cells and isolating the patient's fibroblast culture.

4. Separating the obtained cell material of fibroblasts: sending the 1^{st} part for research, keeping the 2^{nd} part for further modification and introduction to the patient.

5. Analyzing the primary fibroblast culture by determining the DNA sequence of genes and their activity.

5a) analyzing the DNA sequence of these genes, for example, using the sequencing method in order to reveal mutations, i. e. finding out whether the DNA corresponds to the norm.

5b) analyzing the gene expression by measuring the relative quantity of the specific mRNA, i. e. determining the gene activity.

6. Comparing the findings with normal group data, accepting the gene sequences given in the GenBank database, as well as the gene expression data in the UniGene database as the norm.

7. On the basis of the obtained genetic research data, making a conclusion about the connection of the revealed deviations with the patient's skin changes.

8. Performing the actions that allow to compensate for these deviations:

8.1. Creating the genetic constructs with the cDNA of the patient's genes the activity of which is changed, or the DNA structure of which has deviations, and making sure the structure and function of the said genes are like those of the normal genes.

8.2. Embedding these genetic constructs in the patient's fibroblast culture.

Using the genetic construct to embed cDNA in cells in conjunction with dendrimeric macromolecules or liposomes, or amphiphilic block copolymers, which does not exclude other methods for transfection of the genetic construct into the patient's fibroblast culture.

9. Returning the cell culture of modified autogenous fibroblasts bearing the genetic construct to the patient's body.

Introducing to the patient the autogenous fibroblasts modified with the genetic constructs containing the cDNAs of the patient's genes the activity of which is changed, or the DNA structure of which has deviations.

10. Studying the patient's skin parameters after introducing the modified autogenous fibroblasts bearing the created genetic construct.

### BRIEF DESCRIPTION OF THE DRAWINGS

FIG. 1 shows relative reduction of wrinkles (in percent) depending on the time after the introduction of autogenous fibroblasts (Patient 1A):

the curve of the periorbital area,

the curve of the buccal area,

the curve of the peribuccal area.

FIG. 2 shows relative reduction of wrinkles (in percent) depending on the time after the introduction of autogenous fibroblasts (Patient 2A):

the curve of the periorbital area,

the curve of the buccal area,

the curve of the peribuccal area.

FIG. 3 shows relative reduction of wrinkles (in percent) depending on the time after the introduction of autogenous fibroblasts (Patient 3A):

the curve of the periorbital area,

the curve of the buccal area,

the curve of the peribuccal area.

FIG. 4 shows relative reduction of wrinkles (in percent) depending on the time after the introduction of autogenous fibroblasts (Patient 4A):

the curve of the periorbital area,

the curve of the buccal area,

the curve of the peribuccal area.

FIG. 5 shows relative reduction of wrinkles (in percent) depending on the time after the introduction of modified autogenous fibroblasts (Patient 1B):

the curve of the periorbital area,

the curve of the buccal area,

the curve of the peribuccal area.

FIG. 6 shows relative reduction of wrinkles (in percent) depending on the time after the introduction of modified autogenous fibroblasts (Patient 2B):

the curve of the periorbital area,

the curve of the buccal area,

the curve of the peribuccal area.

FIG. 7 shows relative reduction of wrinkles (in percent) depending on the time after the introduction of modified autogenous fibroblasts (Patient 3B):

the curve of the periorbital area,

the curve of the buccal area,

the curve of the peribuccal area.

FIG. 8 shows relative reduction of wrinkles (in percent) depending on the time after the introduction of modified autogenous fibroblasts (Patient 4B):

the curve of the periorbital area,

the curve of the buccal area,

the curve of the peribuccal area.

### DETAILED DESCRIPTION OF THE PREFERRED EMBODIMENTS

In the process of skin aging, caused, first of all, by ultraviolet rays and/or active forms of oxygen, the activity of the TGF-β-dependent signal pathway decreases either due to the reduced quantity of type II receptors for TGF-β (Tβ RII), or due to the reduced activity of the gene TGFB1. This defect leads to the reduction of expression of the connective tissue growth factor (CTGF) and type 1 collagen (COL1A1 and COL1A2), which are regulated by TGF-β. The increase in the quantity of TGFβ RII receptors and the level of the TGF-β protein restores the TGF-β signalling and leads to the rise of the expression of CTGF and type I collagen (Age (Dordr) 2014 Feb. 20; Am. J. Pathol. 2004, 165(3) 741-751).

One more effect in place in the process of aging is the decreasing level of superoxide dismutase (SOD1 and SOD2) and glutathione peroxidase (GPX1 and GPX3), which protect skin fibroblasts (and other cells) from ultraviolet and active forms of oxygen (Aging (Albany N.Y.) 2012, 4(1), 3-12; J Gerontol A Biol Sci Med Sci. 2009, 64, 1114-1125).

The skin aging processes are also affected by a significant reduction of the expression of CLCA2 (Br. J. Dermatol. 2014 Apr. 4).

Accordingly, when conducting research in order to embody the claimed method we selected the following genes:

TGFB1—beta-1-transforming growth factor,

TGFBR2—beta-II-receptor of the transforming growth factor,

COL1A1—alpha-1-collagen type 1,

COL1A2—alpha-2-collagen type 1,

SOD1—solubilized superoxide dismutase-1,

SOD2—solubilized superoxide dismutase-2,

GPX1—glutathione-peroxydase-1,

GPX3—glutathione-peroxydase-3,

CLCA2—chloride channel complexing protein-2

A specialist of any level understands that the genes taken in these examples for the purposes of research and further possible modification do not limit the use of the claimed method for other well-known genes. These genes were selected to prove the claimed method as the ones that are mostly exposed to changes in the process of skin aging.

This invention may be proven by the following embodiments:

### Example 1

The objective of this embodiment is to conduct the functional diagnostics of the skin of a large number of patients in order to select 4 pairs of patients for the further research; the patients in each pair are to be of the same sex, more or less the same age and similar skin aging type, any of the pairs is to have the identified reduction of expression of the same gene, or the same mutant gene variant. This selection is made to introduce the culture of autologous fibroblasts without genetic modifications to one of the pair representatives (the control group), and the fibroblasts modified with genetic constructs as per the claimed invention—to the second patient of the pair.

We selected 200 volunteer patients of both sexes at the age from 38-67 years. We conducted the functional diagnostics of the patient's skin using the measuring instruments that allow obtaining quantitative parameters characterizing the patient's skin condition.

We took some biopsy samples of the skin. We sampled the material in the area not affected by ultraviolet, behind the ear, using the skin biopsy device Epitheasy 3.5 (Medax SRL). We first rinsed the patient's skin with a sterile physiological solution and anesthetized it with a lidocaine solution. The minimal size of the biopsy sample was 3 mm. Then we used the biopsy sample to obtain the primary culture of the patient's fibroblasts. For that purpose we placed the biopsy sample into a sterile Petri dish, rinsed it with the DMEM medium and incubated in 0.25% trypsin solution at the room temperature for 30 minutes. Then we separated the dermis from the epidermis and cut the dermis using the surgical scissors to 3-4 fragments. Then we removed the medium and dried the biopsy sample pieces in the air for 15 minutes to ensure better adhesion. Later, we added the DMEM medium with 10% fetal calf serum and 100 U/ml of ampicillin to the biopsy sample and put the dish into the incubator.

Cells were grown at +37° C. in the environment containing 5% CO₂.

We observed the growth of cells based on the <<halo>> developing around the biopsy pieces. As soon as the cells covered 75% of the surface, we performed one more trypsinization by adding 1 ml of 0.25% trypsin solution to the culture medium. After separating the cells (2 or 3 minutes of incubation at the room temperature) we added 3 ml of the culture medium and resuspended it carefully. Some suspension was used for the immunohistochemical analysis. For that purpose, we rinsed the fibroblast monolayer grown on a glass plate with PBS buffer, fixed it in 4% formaldehyde and treated with monoclonal antibodies to collagen-4 and to filaggrin, as well as polyclonal antibodies to collagen-1 and collagen-3. We stained the preparation with the help of the streptavidin-biotin-peroxydase system with the visualisation by diaminobenzidine.

We were growing the remaining suspension for 5 days in the incubator at +37° C. in the environment containing 5% CO₂. We performed trypsinization and subcultivation every 5 days.

To ensure long-term storage we centrifuged 2 ml of the trypsinized suspension for 5 minutes at 700 rev/min, rinsed the packed cells with the calcium- and magnesium-free medium, centrifuged one more time and resuspended the packed cells in the medium of 30% DMEM, 10% fetal serum and 10% DMSO. We stored the suspension at the temperature of minus 150° C.

One part of the fibroblast culture was sent for the research including the isolation of RNA and DNA with their subsequent analysis. The second part was kept for the further introduction to the patients; we also used this part of fibroblasts for genetic modification prior to the introduction to the patients, in accordance with the claimed invention.

We isolated RNA in order to analyze the level of expression of the following genes: TGFB1, TGFBR2, COL1A1, COL1A2, SOD1, SOD2, GPX1, GPX3 and CLCA2.

We isolated RNA from the suspension containing at least 10⁶ cells. To isolate RNA we used RNeasy Mini Kit (Qiagen). We analyzed the isolated RNA spectrophotometrically by measuring the optic density ratio at 260 and 280 nm, as well as using the capillary electrophoresis in the device QIAxcel (Qiagen) with the cartridge RNA Qiality Control. In our further work we used only those samples for which the total quantity of the isolated RNA was not less than 50 μg of RNA, the ratio D260:D280—not less than 1.8, and the band ratio 28S: 18S in the capillary electrophoresis—not less than 1:1. We synthesized the total cDNA using the reverse transcriptase RevertAid (Fermentas), following the recommendations of the manufacturer. We used 1 to 2 μg of the total RNA as the matrix to synthesize the first cDNA chain. To conduct the reverse transcription we added to the reaction mixture 100 to 200 U of the reverse transcriptase and 10 pmol of the random 9-nucleotide primer.

We analyzed the gene expression level using the well-known methodology described, for example, in [Analytical Biochemistry 2001, 295 (1), 17-21] and in [Nucleic Acids Research 1993, 21(4), 993-998]. To the amplification mixture (the volume of 40 μl) we added 50 ng of the total first chain cDNA, 0.5 μM of each primer, 250 μM of each deoxynucleotide triphosphate, 10 mM of Tris-HCl pH 9, 50 mM of NH₄Cl, 1.5 mM of MgCl₂ and 1 U of Taq-polymerase (Fermentas). We conducted the amplification using the thermal cycler MasterCycler Gradient (Eppendorf). We used the following amplification conditions: the initial denaturation at +94° C. for 3 minutes, then from 25 to 33 cycles including denaturation at +94° C. for 30 seconds, the annealing of primers at +57° C. for 30 seconds and elongation at +72° C. for 1 minute. At the end of the cycles, we conducted the final elongation at +72° C. for 5 minutes.

When assessing the activity of these genes, we used as the measure of control the constitutive genes the level of expression of which in fibroblasts is comparable to that of normal genes under study. The data on the expression of normal genes were taken from the UniGene database (www.ncbi.nlm.nih.gov/UniGene).

The level of expression of the genes TGFB1, SOD2 and CLCA2 was compared with that of the gene TPMT.

the level of expression of the gene TGFBR2—with that of PRKAG;

the level of expression of the gene COL1A1—with that of PGK1;

the level of expression of the genes SOD1 and GPX1—with that of HADHA;

the level of expression of the gene COL1A2—with that of UQCRC1:

the level of expression of the genes SOD1 and GPX1—with that of HADHA;

the level of expression of the gene GPX3—with that of SGSH;

We analyzed the amplification products by conducting the capillary electrophoresis using the capillary electrophoresis system QIAxcel (Qiagen). We used the DNA High Resolution cartridge that allows determining the length of amplification products with the accuracy of up to 3 nucleotide pairs. We used the marker QX DNA Size Marker 50 bp-1.5 kb (Qiagen). We analyzed the gel in order to determine the length and the concentration of the amplification products using BioCalculator Software v 2.0 (Qiagen).

We isolated the DNA to identify the mutations affecting the gene activity and/or the activity of the protein coded by this gene. We analyzed the primary DNA structure using the pyrosequencing method. We analyzed those mutations the functional meaning of which is described in literature.

We isolated the DNA for the genetic typing with the help of the QIAamp DNA Mini Kit (Qiagen) in accordance with the instruction supplied with the kit, using the methodology of DNA isolation from the cell culture. We determined the DNA concentration spectrophotometrically by analyzing the optic density spectrum from 320 to 240 nm.

To perform the genetic typing we amplified the DNA areas containing the mutations being analyzed from specific PCR-primers. For the purpose of amplification, we used the GenePak PCR Core kit (manufactured by <<Isogen Laboratory>> LLC, the Russian Federation) in accordance with the instruction supplied with the kit. We added 50 ng of the DNA and 1 μM of each primer to the amplification mixture. We used the following amplification conditions: the initial denaturation at +94° C. for 3 minutes, then 37 cycles including denaturation at +94° C. for 30 seconds, the annealing of primers at +55° C. for 30 seconds and elongation at +72° C. for 1 minute. At the end of the cycles, we conducted the final elongation at +72° C. for 3 minutes.

We analyzed the amplification products in order to reveal mutations using the pyrosequencing method in the device PyroMark Q96ID (Qiagen).

The DNA polymorphisms being analyzed are listed in Table 1.

TABLE 1

| Gene | Polymorphism | Risk allele |
|---|---|---|
| TGFB1 | rsI800471 (Arg25Pro) | C (Pro) |
| TGFB1 | rs1800469 (−509T > C) | C |
| TGFB1 | rs2241712 | A |
| TGFB1 | r51800470 (869T > C) | T |
| COL1A1 | rs18000012 (1245G > T) | T |
| COL1A1 | rs1107946 (Sp1) | T |
| COL1A1 | rs2412298 (−1663 InsDelT) | deletion |
| COL1A2 | rs3216902 | deletion |
| SOD2 | rs4880 (Val16Ala) | C |
| GPX1 | rs1050450 (Pro200Leu) | T (Leu) |
| GPX1 | rs1800668 | T |

We selected 4 pairs of patients based on the findings. Each pair comprised patients of the same sex, more or less the same age (the difference did not exceed 4 years), with the similar skin aging type. The patients making up one pair had the reduced expression of the same gene expressed to the similar extent. One of the pairs also included the patients with the same mutant variant of the gene TGFB1 [variant C (Pro) in the polymorphism area rs1800471 (G/C; Arg25Pro)].

The data on the pairs selected on the basis of the above principle are provided in Table 2.

TABLE 2

| Pairs of | | | | Gene with reduced | Revealed |
|---|---|---|---|---|---|
| patients | Sex | Age | Aging type | expression | mutations |
| 1 | Female | 46-48 | Tired | TGFBR2; COL1A1; | — |
| | | | | COL1A2 | |
| 2 | Female | 53-55 | Small lines | TGFB1; COL1A1; | rs1800471 |
| | | | | COL1A2 | |
| 3 | Female | 65-68 | Deforming | COL1A1 | |
| 4 | Male | 58-61 | Deforming | CO1A1 | |

### Example 2

The culture of autogenous fibroblasts without genetic modifications was injected to one of the patients of each pair (these patients were marked with the letter A). For that purpose, we centrifuged the second (stored) part of the cell culture one more time at 700 rev/min, rinsed the cells twice with a physiological solution, resuspended them in a physiological solution in the proportion of 5 mln cells in 1 ml and used them to introduce to one patient of each pair marked A, in accordance with the methodology described below. A similar methodology is described in the Aesthetic Medicine Bulletin, 2011, 10(2), 16-26). We injected the cells suspended in a physiological solution, for example, using the tunnel method with the help of 30 G needles 13 mm long to the depth of 3 mm in the area of facial skin wrinkles. The total single doze did not exceed 15 mln cells (3 ml of the suspension). The fibroblast suspension was injected 3 times—4 and 8 weeks after the first introduction. Topographic peculiarities of the patients' skin surface were examined prior to all the procedures, on week 4 and week 8 prior to the injection, as well as 12 weeks following the first injection of fibroblasts using the laser profilometry method PRIMOS (Phase (shift) Rapid In Vivo Measurement Of Skin) with the resolution of 0.004 mm. We also calculated the clinical aging index CAI for the face and the neck based on 11 signs (Clinical Aging Index, R. Bazin):

Main signs of aging:

forehead wrinkles, nasolabial fold, depth of <<crow's feet>>, quantity of <<crow's feet>>, wrinkles in the corners of the mouth, upper lip wrinkles, eye-bags, wrinkles under the eyes, glabellar wrinkles, lower face ptosis, circular neck folds.

The results of the profilometry and the calculation of the aging index for the patients of Group A are provided in Table 3.

TABLE 3

| | | Pair | | | |
|---|---|---|---|---|---|
| | | 1 | 2 | 3 | 4 |
| | | Patient | | | |
| | | 1A | 2A | 3A | 4A |
| | | Sex | | | |
| | | Female | Female | Female | Male |
| | | Age | | | |
| | | 46 | 53 | 65 | 58 |
| Prior to | Average | Periorbital area - | Periorbital area - | Periorbital area - | Periorbital area - |
| treatment | profilometric | 16.24; buccal | 16.29; buccal | 23.88; buccal | 21.43; buccal |
| | values, mm | area - 11.22; | area - 14.42; | area - 14.55; | area - 12.22; |
| | | perioral area - | perioral area - | perioral area - | perioral area - |
| | | 6.84 | 6.92 | 7.16 | 7.25 |
| | Aging index | 1.116 + 0.967 | 1.278 + 0.226 | 1.976 + 0.267 | 2.982 + 0.378 |
| After 4 | Average | Periorbital area - | Periorbital area - | Periorbital area - | Periorbital area - |
| weeks | profilometric | 16.20; buccal | 16.21; buccal | 23.72; buccal | 21.27; buccal |
| | values, mm | area - 11.16; | area - 14.32; | area - 14.42; | area - 12.04; |
| | | perioral area - | perioral area - | perioral area - | perioral area - |
| | | 6.81 | 6.83 | 7.04 | 7.16 |
| | Aging index | 1.116 + 0.001 | 1.278 + 0.002 | 1.976 + 0.009 | 2.982 + 0.009 |
| After 8 | Average | Periorbital area - | Periorbital area - | Periorbital area - | Periorbital area - |
| weeks | profilometric | 16.06; buccal | 16.17; buccal | 23.56; buccal | 21.19; buccal |
| | values, mm | area - 11.04; | area - 14.28; | area - 14.37; | area - 11.88; |
| | | perioral area - | perioral area - | perioral area - | perioral area - |
| | | 6.73 | 6.82 | 6.95 | 7.11 |
| | Aging index | 1.13 + 0.003 | 1.276 + 0.67 | 1.959 + 0.06 | 2.973 + 0.06 |
| After 12 | Average | Periorbital area - | Periorbital area - | Periorbital area - | Periorbital area - |
| weeks | profilometric | 15.78; buccal | 16.15; buccal | 23.51; buccal | 21.12; buccal |
| | values, mm | area -10.96; | area - 14.28; | area - 14.32; | area - 11.79; |
| | | perioral area - | perioral area - | perioral area - | perioral area - |
| | | 6.61 | 6.81 | 6.89 | 7.04 |
| | Aging index | 1.13 + 0.05 | 1.269 + 0.03 | 1.955 + 0.09 | 2.975 + 0.05 |

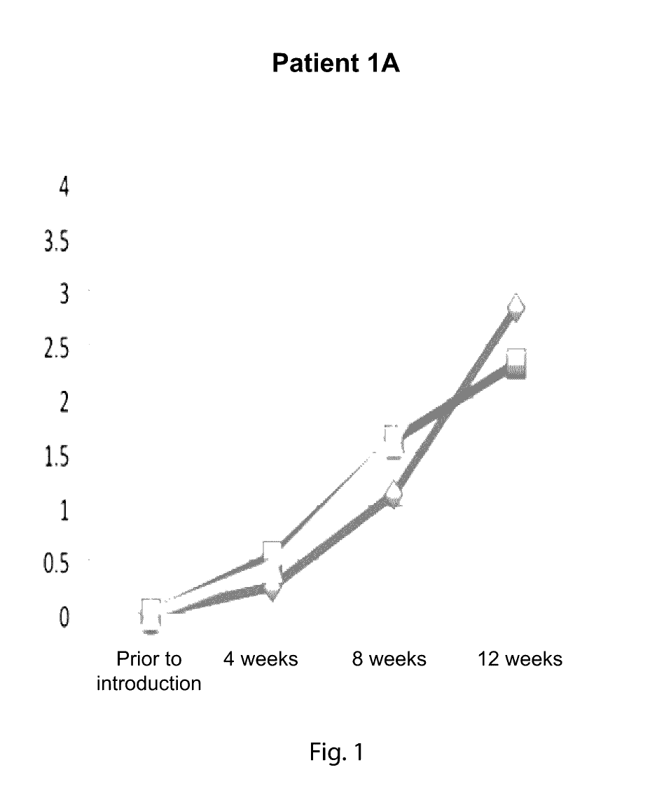

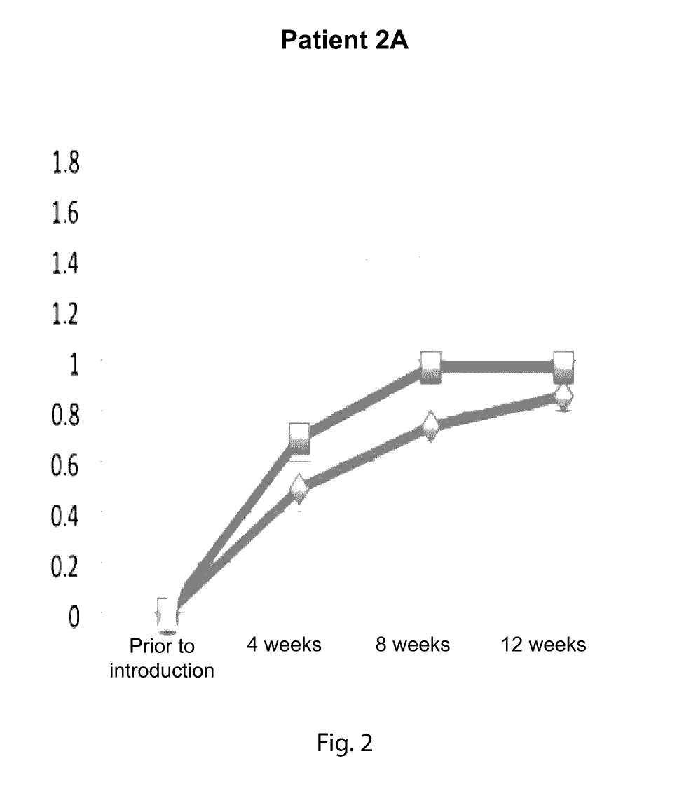

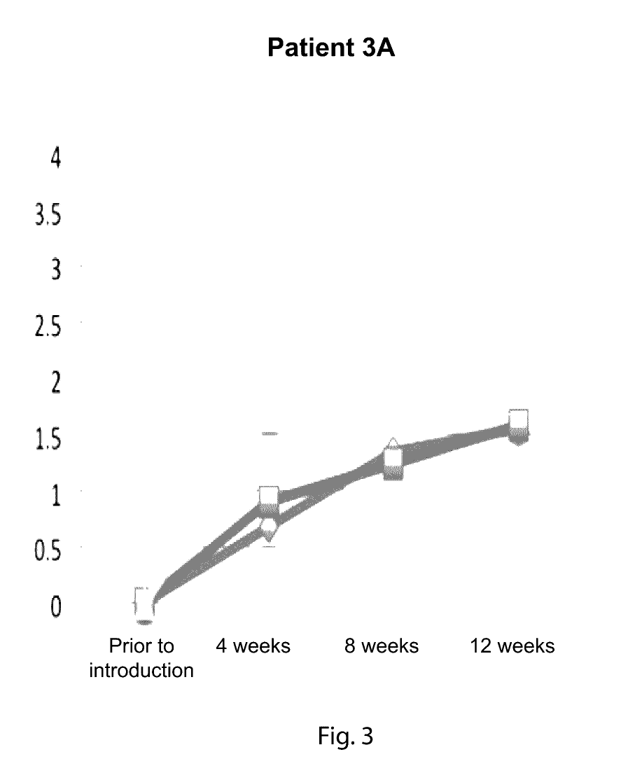

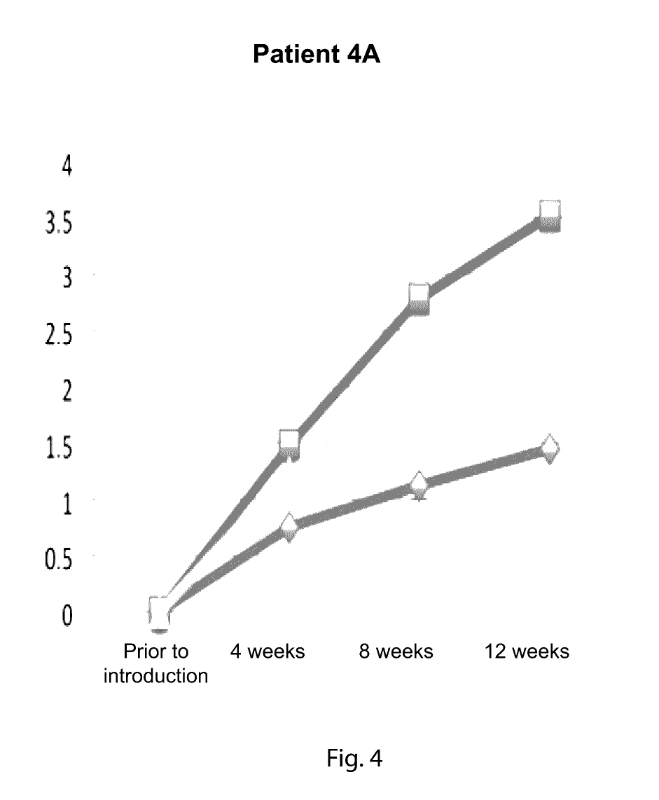

Relative reduction of wrinkles for the patients 1A, 2A, 3A, 4A after the introduction of autologous fibroblasts is shown in FIGS. 1-4.

Table 4 contains the data of assessing the facial skin parameters obtained in the course of questioning and examining the patients.

TABLE 4

| | | No. | | | |
|---|---|---|---|---|---|
| | | 1A | 2A | 3A | 4A |
| | | Age | | | |
| | | 46 | 53 | 65 | 58 |
| | | Sex | | | |
| | | Female | Female | Female | Male |
| Texture | 4 weeks | 0 | − | − | − |
| | 8 weeks | 0 | − | 0 | 0 |
| | 12 weeks | 0 | − | 0 | 0 |
| Moisture | 4 weeks | − | 0 | − | − |
| | 8 weeks | − | 0 | + | + |
| | 12 weeks | + | 0 | + | + |
| Elasticity | 4 weeks | 0 | − | 0 | − |
| | 8 weeks | 0 | 0 | 0 | − |
| | 12 weeks | + | + | 0 | 0 |
| Swelling | 4 weeks | − | − | − | + |
| | 8 weeks | − | 0 | 0 | + |
| | 12 weeks | 0 | 0 | 0 | + |
| Vascular pattern | 4 weeks | 0 | − | 0 | − |
| | 8 weeks | 0 | 0 | 0 | 0 |
| | 12 weeks | 0 | 0 | 0 | 0 |

Signs in the Table:
“+” stands for improvement
“−” stands for deterioration
“++” stands for significant improvement
“−−” stands for significant deterioration
“0” stands for no dynamics

All the patients subjectively felt the increased overall tonus and performance capability after the first injection. During 48 hours following the first injection of fibroblasts, the patients had low-grade fever with no overlay of infectious diseases.

The data provided in Table 3 imply that the introduction of autologous fibroblasts without any additional modifications did not result in any objective significant facial skin changes that may be detected with the help of the aging index and profilometry.

### Example 3

The autogenous fibroblasts modified with the genetic constructs containing the cDNA of the gene the activity of which was changed, or the primary DNA structure of which had deviations were injected to every second patient of each pair (these patients were further marked with the letter B).

Description of the overall sequence of actions for the patients of Group B.

Based on the obtained genetic research data we made a conclusion about the connection of the revealed deviations in the gene structure and/or function with the patient's skin changes.

We performed actions that enabled compensate for these deviations: we synthesized the cDNAs of these genes and placed these cDNAs in a vector construct for the further transfection into the cells.

This invention uses the method for correcting genetic defects, which implies that the genetic information injected to fibroblasts is not included in the cell genome.

To reach the efficient expression we placed the target gene in the vector construct, where it had to stay under control of the regulatory elements operating independently of the nuclear apparatus (Int J Pharm 2001, 229, 1-21; Hum Gene Ther 1997, 8, 1763-1772; Anesth. Analg. 2001, 92, 19-25; Journal of Gene Medicine 2001, 3, 384-393; Human Gene Therapy 2000, 11, 2253-2259; Human Gene Therapy 1996, 7, 1205-1217). The lifetime of such constructs in the cell may be measured in weeks, but the effect appears to be pronounced and long.

To introduce the obtained constructs to the cell we used the method that did not demand any additional involvement of viral particles. In one instance, we used auxiliary molecules called dendrimers [Chem. Rev. 2009, 109, 3141-3157; Mol. Pharmaceutics 2012, 9, 341; PNAS 1996, 93, 4897-4902; US 20120045430; EP 2543659 A1]. A dendrimer is a macromolecule with the ramified structure and charged groups on the surface facilitating its penetration through the cell membrane. The DNA forms a complex with the macromolecule, the complex passes through the cytoplasmic membrane of the cell, and the genetic construct appears in the cytoplasm. The efficacy of using the dendrimer complex with the genetic construct not requiring the embedding in the genome is comparable to the wide-spread method for transfection implying the embedding of the injected genetic information in the cell genome. This method, as a rule, demands the use of vector constructs based on viruses, i. e. the adenovirus, the adeno-associated virus or the retrovirus (Lancet 2007, 369(9579), 2097-2105; Science 2000, 288(5466), 669-672; Cancer Gene Ther 2007, 14, 599-615; U.S. Pat. No. 6,461,606 B1). The methodology using virus constructs may be present a potential biological hazard as it requires the presence of virus proteins, which may specifically cause the immune response (Ace Chem Res 1993, 26, 274-278; Curr Opin Biotechnol 1993, 4, 705-710; Science 2000, 286, 2244-2245). However, the use of virus constructs for the embodiment of this invention is not excluded. Instead of dendrimers, in our experiments we also used liposomes, i. e. macromolecular phospholipid complexes produced in aqueous solutions and capable of interacting with macromolecules (for example, nucleic acids) and transporting them to the cell (a similar method is described in Gene Therapy, 5(3), 380-387 (1998)). To introduce genetic constructs into the cell we also used amphiphilic block copolymers, including the hydrophilic and hydrophobic polymeric blocks, which are also capable of interacting with macromolecules (for example, nucleic acids) and transporting them to the cell (Cell Transplantology and Tissue Engineering, 2012, 7(3), 101-104; Int J Pharm, 2012, 427, 80-87).

We embedded the created genetic construct in the patient's cells, for example, by introducing the gene material complex with dendrimeric macromolecules, liposomes or amphiphilic block copolymers or using another well-known not described method, which is apparent for a specialist of any level.

To introduce the obtained constructs to the cell in this research we used the method that did not demand any additional involvement of viral particles. In one instance, we used polyamidoamine dendrimers (Sigma-Aldrich). To increase the stability we brought the pH factor in the aqueous dendrimer solution to 7.4. We also dissolved the plasmid-based construct in deionized water and incubated for 30 minutes with the dendrimer solution using the plasmid:dendrimer mass ratio of 1:2. We assessed the efficacy of the dendrimer and plasmid binding based on the retardation of the DNA-dendrimer complex migration in 1% agarose gel. Then we added the solution containing 5 to 10 μg of the DNA-dendrimer complex in the DMEM medium with 10% fetal serum and ampicillin to the cells and grew them in this medium for 72 hours.

We also used the constructs called liposomes to transport the plasmid to the cells. In this case we used a lipid-solubilized complex consisting of cationic lipid and beta-cyclodextrin. We applied the well-known methodology described in (Gene Therapy, 5(3), 380-387 (1998)) with some changes. We mixed cholesterol dissolved in methyl-beta-cyclodextrin with the cationic lipid DOTAP (N-[1-(2,3-Dioleoiloxi)]-N,N,N-trimethylammonium propane) in the proportion of 2:1, brought pH to 8.0 with 20 mM of HEPES buffer and incubated at the room temperature for 15 minutes (all reagents were Sigma-Aldrich). Then we added the DNA solution in deionized water, the final mass ratio DNA:DOTAP:cholesterol was 1:2:4, and incubated the mixture at the room temperature for 30 minutes. We added the mixture for transfection (the final volume of 150 μl) to the cell suspension in 1 ml of the culture medium DMEM with 10% fetal serum and ampicillin and grew the cells in this medium for 72 hours.

We also used amphiphilic block copolymers to transport the plasmid to the cells. We applied the well-known methodology described, for example, in (Int J Pharm, 2012, 427, 80-87) or in (Macromol. Biosci. 2011, 11, 652-661), with some changes. We synthesized the block copolymer from the mixture of the linear polyethyleneimine (PEI) (Polyscience Inc., USA) and the biofunctional polyethyleneglycol (PEG) N-hydroxysuccinimidyl-75-N-(3-maleimidopropionyl)-amido-4, 7, 10, 13, 16, 19, 22, 25, 28, 31, 34, 37, 40, 43, 46, 49, 52, 55, 58, 61, 64, 67, 70, 73-tetracosaoxapenta-heptacontanoat (MAL-dPEG™-NHS ester, Quanta BioDesign, Ltd., USA) in borate buffer. We prepared the polyplex 1 hour before the introduction to the cells by mixing the block copolymer solution with the DNA. We added the mixture for transfection to the cell suspension in 1 ml of the culture medium DMEM with 10% fetal serum and ampicillin and grew the cells in this medium for 72 hours.

Then we returned the cell culture bearing the genetic construct, i. e. the modified autogenous fibroblasts, to the patient's body.

We studied the patient's skin parameters following the therapy course that comprised the introduction of autologous fibroblasts bearing the created genetic construct to the patient.

We studied the patients' condition after the introduction of autologous fibroblasts bearing the created genetic construct to such patients.

We assessed the dermatologic status of all the patients prior to the introduction of the modified fibroblasts. To study the efficacy of the treatment we tested the patients using special instrumental procedures of functional diagnostics. We conducted the testing prior to the treatment course, as well as 4, 8 and 12 weeks after the procedure, and this testing included the following methods:

1) the optic profilometry PRIMOS (Phase (shift) Rapid In Vivo Measurement Of Skin) with the resolution of 0.004 mm;

2) the calculation of the clinical aging index CAI for the face and the neck (in accordance with the atlas by R. Bazin) based on 11 signs.

### Example 4

Sequences of Actions for Patient 1B.

Patient 1B of Pair 1, female, 48 years old. Aging type—tired (see Table 2).

During the examination we found: reduced skin tightness, facial swelling, changes in the tonus of facial mimic muscles, intensity of nasolabial folds, eyelid and lip drooping making the impression of tiredness, weariness.

The clinical aging index CAI (the atlas by R. Bazin) prior to the introduction of autologous fibroblasts to the patient was 1.16+0.967, which is indicative of the low degree of intensity of involutional changes.

When analyzing the activity of the above listed genes we discovered that the expression level of the gene TGFBR2 decreased 2.5 times versus the expected one (in relation to the expression of the control gene PRKAG). Furthermore, we discovered that the level of expression of the genes COL1A1 and COL1A2 decreased 2 and 2.2 times, respectively (in relation to the control genes PGK1 and UQCRC1). During the genetic typing, we found only wild-type alleles. Since the level of the protein coded by the gene TGFBR2 breaks down the signalling involving TGF-b and thus affects the activity of the genes COL1A1 and COL1A2, we admitted that the decreased level of expression of the genes COL1A1 and COL1A2 resulted from the reduced expression of the gene TGFBR2. Therefore, we decided to transfect the fibroblast culture of Patient 1B with the genetic construct containing the cDNA of the gene TGFBR2.

For that purpose, we constructed the plasmid pAAV-TGFBR2: we placed the cDNA corresponding to the protein-coding region of the gene TGFBR2 in the vector plasmid pAAV-MCS under control of the promotor CMV [Clin. Med. J. (Engl) 2004, 117(4), 562-565; Gene 1999, 238(2), 397-405]. Then we transfected the plasmid into the patient's autologous fibroblast culture. We performed the transfection using the 5^{th} generation polyamidoamine dendrimers (PAMAM-dendrimers with ethylene diamine surface constructs, Sigma-Aldrich) [Pharm. Sci. Tecnol. Today 2000, 3(7), 232-245; Nanomedicine 2009, 5(3) 287-297]. Following the transfection, we cultured fibroblasts for 72 hours more and then injected the cell suspension to the patient.

We injected the modified autogenous fibroblasts to the patient using the tunnel method along the wrinkle lines with the help of a 30 G needle 13 mm long to the depth of 3 mm with the application of the meso therapy method. The concentration of the modified autogenous fibroblasts in the injected suspension was approximately 5 mln cells in 1 ml of the suspension, the dose of the injected cells did not exceed 15 mln. The suspension of fibroblasts was injected 3 times—4 and 8 weeks after the first introduction. Prior to all the procedures, on week 4 and week 8 prior to the injection, as well as 12 weeks after the first injection of the modified autogenous fibroblasts, we examined the patient using the laser profilometry method. We also calculated the clinical aging index CAI for the face and the neck.

The profilometric values of Patient 1B after the injections of the modified autogenous fibroblasts are provided in Table 5.

TABLE 5

| | Average profilometric values, mm | | |
|---|---|---|---|
| | Periorbital area | Buccal area | Perioral area |
| Prior to introduction | 15.82 | 11.12 | 6.57 |
| 4 weeks | 15.14 | 10.74 | 5.90 |
| 8 weeks | 13.08 | 10.13 | 4.77 |
| 12 weeks | 12.58 | 9.45 | 4.56 |

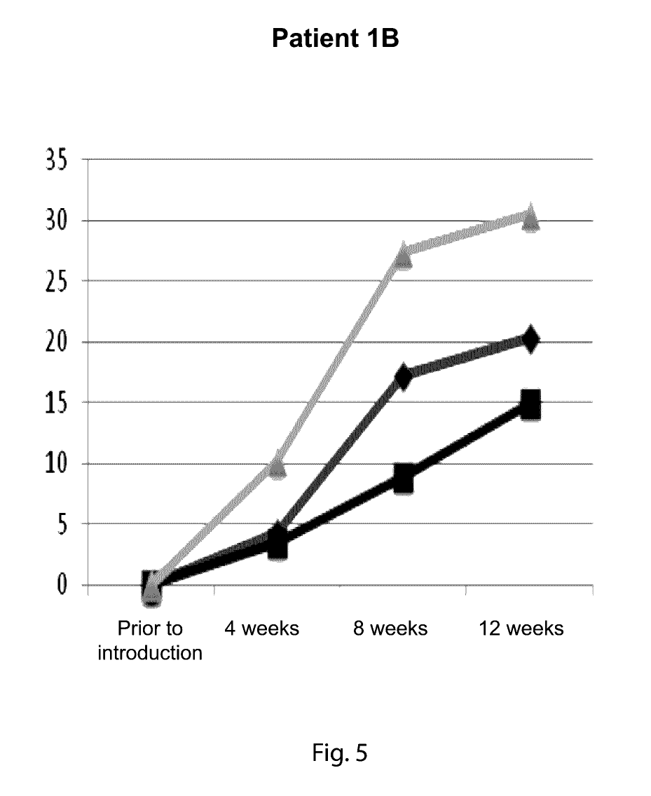

Relative reduction of wrinkles of Patient 1B after the introduction of the modified autogenous fibroblasts is shown in FIG. 5.

The clinical aging index CAI (the atlas by R. Bazin) after 12 weeks was 1.017+0.5670.

### Example 5

Sequences of Actions for Patient 2B.

Patient 2B of Pair 2, female, 55 years old. Small lines aging type. In the anamnesis—COPD.

The clinical aging index CAI (in accordance with the atlas by R. Bazin) prior to the introduction of fibroblasts was 1.278+0.226.

The skin is thinned, dry, prone to irritation and erubescence. The subcutaneous fat is poorly developed, and the muscle tone decrease is not significant, therefore the sagging of facial soft tissues is marked feebly.

When analyzing the expression of the above listed genes we discovered that the expression level of the gene TGFB1 decreased 3 times versus the expected one (in relation to the expression of the control gene TPMT). Furthermore, we discovered that the level of expression of the genes COL1A1 and COL1A2 decreased 1.8 and 2 times, respectively (in relation to the control genes PGK1 and UQCRC1). During the genetic typing, we discovered the variant C (Pro) in the area of the polymorphism rs1800471 in the coding region of the gene TGFB1. No polymorphisms were found in the genes COL1A1 and COL1A2. Since the activity of the gene TGFB1 also affects the activity of the genes COL1A1 and COL1A2, we admitted that the decreased level of expression of the genes COL1A1 and COL1A2 resulted from the reduced expression of the gene TGFB1. COPD in the patient's anamnesis is also an indirect indicator of the abnormal level of synthesis of type 1 collagens. Therefore, we decided to transfect the fibroblast culture of Patient 2B with the genetic construct containing the cDNA of the gene TGFB1 bearing in position 25 the codon CGC coding the remainder of arginine.

For that purpose, we constructed the plasmid pAAV-TGFBI: we placed the cDNA corresponding to the protein-coding region of the gene TGFB1 in the vector plasmid pAAV-MCS under control of the promotor CMV [Clin. Med. J. (Engl) 2004, 117(4), 562-565; Gene 1999, 238(2), 397-405). Then we transfected the plasmid into the patient's autologous fibroblast culture. To transport the plasmid to the cells we used the lipid-solubilized complex comprising the cationic lipid and beta-cyclodextrin [Gene Therapy 1998, 5(3), 380-387]. We mixed cholesterol dissolved in methyl-beta-cyclodextrin with the cationic lipid DOTAP (N-[1-(2,3-Dioleoiloxi)]-N,N,N-trimethylammonium propane) in the proportion of 2:1, brought pH to 8.0 with 20 mM of HEPES buffer and incubated at the room temperature for 15 minutes (all reagents were Sigma-Aldrich). Then we added the DNA solution in deionized water; the final mass ratio DNA:DOTAP:cholesterol was 1:2:4, and incubated the mixture at the room temperature for 30 minutes.

We added the mixture for transfection (the final volume of 150 μl) to the cell suspension in 1 ml of the culture medium DMEM with 10% fetal serum and ampicillin and grew the cells in this medium for 72 hours.

We injected the modified autogenous fibroblasts to the patient using the tunnel method along the wrinkle lines with the help of a 30 G needle 13 mm long to the depth of 3 mm with the application of the meso therapy method. The concentration of the modified autogenous fibroblasts in the injected suspension was approximately 5 mln cells in 1 ml of the suspension, the dose of injected cells did not exceed 15 mln. The suspended modified autogenous fibroblasts were injected 3 times—4 and 8 weeks after the first introduction. Prior to all the procedures, on week 4 and week 8 prior to the injection, as well as 12 weeks after the first injection of fibroblasts, we examined the patient using the laser profilometry method. We also calculated the clinical aging index CAI for the face and the neck.

After the first introduction of the material, the patient subjectively felt the facial skin tension without any apparent changes within the first 48 hours.

There was no increase in arterial blood pressure or other changes associated with the inflammatory process observed.

The profilometric values of Patient 2B after the injections of the modified autogenous fibroblasts are provided in Table 6.

TABLE 6

| | Average profilometric values, mm | | |
|---|---|---|---|
| | Periorbital area | Buccal area | Perioral area |
| Prior to introduction | 16.27 | 13.67 | 6.87 |
| 4 weeks | 15.04 | 13.22 | 4.17 |
| 8 weeks | 13.31 | 11.45 | 3.81 |
| 12 weeks | 12.33 | 10.35 | 3.44 |

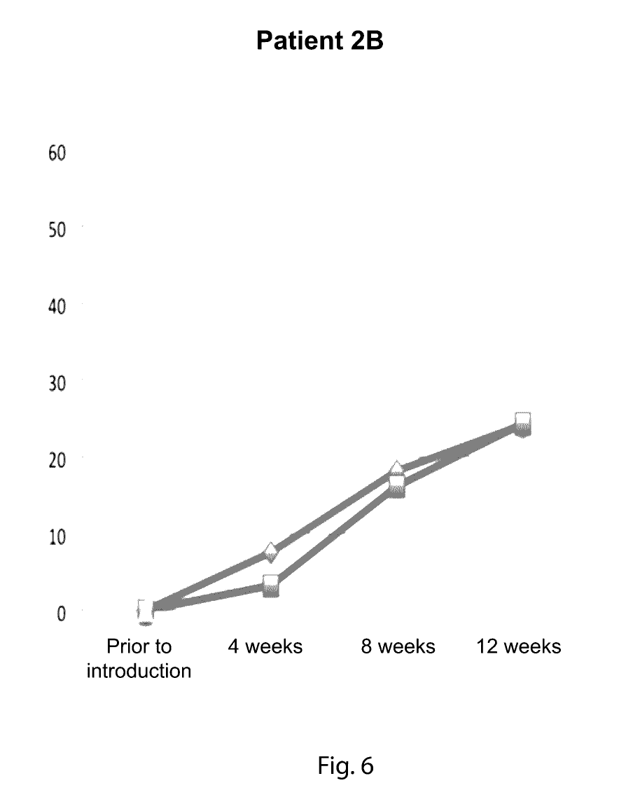

Relative reduction of wrinkles of Patient 2B after the introduction of the modified autogenous fibroblasts is shown in FIG. 6.

The aging index CAI changed to a lesser extent versus those of other patients of Group B, and 12 weeks after the 1^{st} injection it was 1.219+0.023, which is indicative of a significant degree of intensity of involutional changes.

### Example 6

Sequences of Actions for Patient 3B.

Patient 3B of Pair 3, female, 68 years old. Deforming aging type (or large wrinkles).

It is characterized by the skin elasticity deterioration, decreased tone of facial muscles, lymphatic outflow deterioration, as well as venous stasis. The changes in the tone of facial muscles include the hypertonia of the main muscles of the upper and lower thirds of the face and the hypotonia of the muscles mainly of the mid face. We observed the changed configuration of the face and the neck: deformed facial contours, drooping upper and lower eyelids, the development of <<buccula>>, the development of deep folds and wrinkles (nasolabial fold, nuchal-mental fold, wrinkles from the mouth corners to the chin etc.). The patient has well-developed subcutaneous fat. Against the background of the abnormal tone of muscles and the increased stretchability of tissues, we observed the gravitational displacement of the subcutaneous fat in the area of the cheeks with the development of cheek sagging and a so-called <<hernia>> of the lower eyelid constituting the accumulation of fat in the said area.

The clinical aging index CAI prior to the introduction of fibroblasts was 1.976+0.267.

When analyzing the expression of the above listed genes we discovered that the expression level of the gene COL1A1 decreased 3.5 times versus the expected one (in relation to the expression of the control gene PGK1). The expression level of the other analyzed genes remained practically unchanged. During the genetic typing of COL1A1, we did not discover any risk alleles at the points of analyzed polymorphisms. Therefore, we transfected the fibroblast culture of Patient 3B with the genetic construct containing the cDNA of the gene COL1A1.

For that purpose, we constructed the plasmid pAAV-COL1A1: we placed the cDNA corresponding to the protein-coding region of the gene COL1A1 in the vector plasmid pAAV-MCS under control of the promotor CMV [Clin. Med. J. (Engl) 2004, 117(4), 562-565; Gene 1999, 238(2), 397-405]. Then we transfected the plasmid into the patient's autologous fibroblast culture. We performed the transfection using amphiphilic block copolymers. We synthesized the block copolymer from the mixture of the linear polyethyleneimine (PEI) (Polyscience Inc., USA) and the biofunctional polyethyleneglycol (PEG) N-hydroxysuccinimidyl-75-N-(3-maleimidopropionyl)-amido-4,7,10,13,16,19,22,25,28,31,34,37,40,43,46,49,52,55,58,61,64,67,70,73-tetracosaoxapenta-heptacontanoat (MAL-dPEG™-NHS ester, Quanta BioDesign, Ltd., USA) in borate buffer. We prepared the polyplex 1 hour before the introduction to the cells by mixing the block copolymer solution with the DNA in the proportion of 55 μM of the block copolymer: 150 μg of the plasmid DNA. The final concentration of the block copolymer was 10 μM. We added 1 ml of this solution to 2 ml of the cell suspension in the culture medium, grew the cells in this medium for 72 hours and then injected them to the patient.

We injected the modified autogenous fibroblasts to the patient using the tunnel method along the wrinkle lines with the help of a 30 G needle 13 mm long to the depth of 3 mm with the application of the meso therapy method. The concentration of the modified autogenous fibroblasts in the injected suspension was approximately 5 mln cells in 1 ml of the suspension, the dose of injected cells did not exceed 15 mln. The suspended modified autogenous fibroblasts were injected 3 times—4 and 8 weeks after the first introduction. Prior to all the procedures, on week 4 and week 8 prior to the injection, as well as 12 weeks after the first injection of fibroblasts, we examined the patient using the laser profilometry method. We also calculated the clinical aging index CAI for the face and the neck.

Patient 3B, like Patient 1B, subjectively felt some shiver, although there were no objective data confirming the temperature rise, subjectively felt the facial skin tension without any apparent changes within the first 48 hours.

The profilometric values of Patient 3B after the injections of the modified autogenous fibroblasts are provided in Table 7.

TABLE 7

| | Average profilometric values, mm | | |
|---|---|---|---|
| | Periorbital area | Buccal area | Perioral area |
| Prior to introduction | 23.73 | 14.47 | 7.15 |
| 4 weeks | 21.79 | 14.01 | 5.29 |
| 8 weeks | 18.12 | 13.82 | 4.08 |
| 12 weeks | 15.96 | 13.71 | 3.55 |

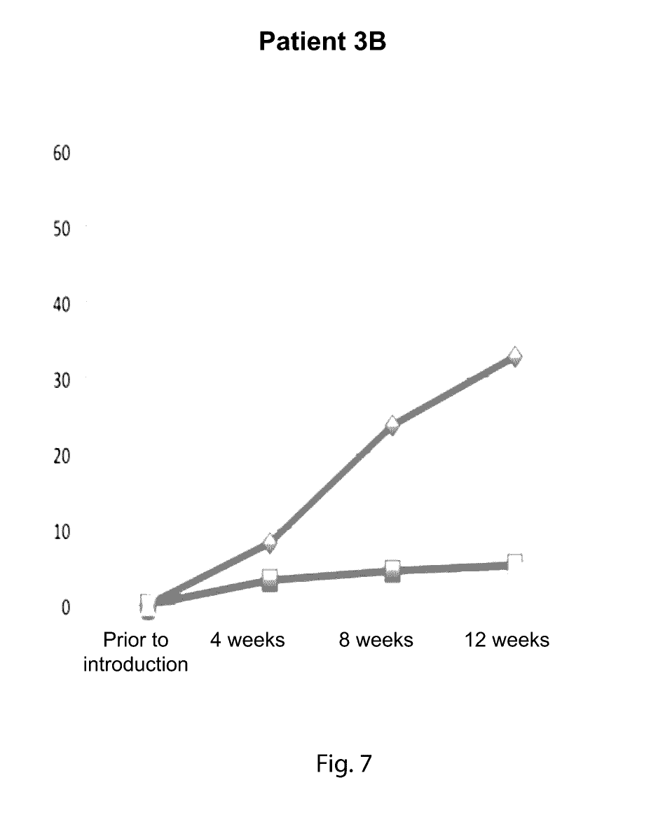

Relative reduction of wrinkles of Patient 3B after the introduction of the modified autogenous fibroblasts is shown in FIG. 7.

The clinical aging index CAI after 12 weeks was 1.5056+0.4726.

### Example 7

Sequences of Actions for Patient 4B.

Patient 4B of Pair 4, male, 61 years old. Deforming aging type. Face puffiness, pitted acne-cicatrical changes on the lower face third, highly intensive couperosis cutaneous changes.

The clinical aging index CAI prior to the introduction of fibroblasts was 2.982+0.238.

When analyzing the expression of the above listed genes we discovered that the expression level of the gene COL1A1 decreased 2.5 times versus the expected one (in relation to the expression of the control gene PGK1). The expression level of the other analyzed genes remained practically unchanged. During the genetic typing of COL1A1, we did not discover any risk alleles at the points of analyzed polymorphisms. Therefore, we transfected the fibroblast culture of Patient 4B with the genetic construct containing the cDNA of the gene COL1A1.

For that purpose, we constructed the plasmid pAAV-COL1A1: we placed the cDNA corresponding to the protein-coding region of the gene COL1A1 in the vector plasmid pAAV-MCS under control of the promotor CMV [Clin. Med. J. (Engl) 2004, 117(4), 562-565; Gene 1999, 238(2), 397-405]. Then we transfected the plasmid into the patient's autologous fibroblast culture. We performed the transfection using the 5^{th} generation polyamidoamine dendrimers (PAMAM-dendrimers with ethylene diamine surface constructs, Sigma-Aldrich #536709) [Pharm. Sci. Tecnol. Today 2000, 3(7), 232-245; Nanomedicine 2009, 5(3) 287-297]. To increase the stability we brought the pH factor of the aqueous dendrimer solution to 7.4. We also dissolved the plasmid-based construct in deionized water and incubated for 30 minutes with the dendrimer solution using the plasmid:dendrimer mass ratio of 1:2. Then we added the solution containing 5 to 10 μg of the DNA-dendrimer complex in the culture medium to the cells, grew the cells in this medium for 72 hours and then injected them to the patient.

We injected the modified autogenous fibroblasts to the patient using the tunnel method along the wrinkle lines with the help of a 30 G needle 13 mm long to the depth of 3 mm with the application of the meso therapy method. The concentration of the modified autogenous fibroblasts in the injected suspension was approximately 5 mln cells in 1 ml of the suspension, the dose of the injected cells did not exceed 15 mln. The suspension of the modified autogenous fibroblasts was injected 3 times—4 and 8 weeks after the first introduction. Prior to all the procedures, on week 4 and week 8 prior to the injection, as well as 12 weeks after the first injection of the modified autogenous fibroblasts, we examined the patient using the laser profilometry method. We also calculated the clinical aging index CAI for the face and the neck.

The patient did not have any subjective bad feelings. The profilometric values of Patient 4B after the injections of the modified autogenous fibroblasts are provided in Table 8.

TABLE 8

| | Average profilometric values, mm | | |
|---|---|---|---|
| | Periorbital area | Buccal area | Perioral area |
| Prior to introduction | 21.36 | 12.19 | 7.18 |
| 4 weeks | 20.67 | 11.93 | 6.01 |
| 8 weeks | 16.75 | 11.90 | 4.28 |
| 12 weeks | 15.83 | 11.88 | 4.16 |

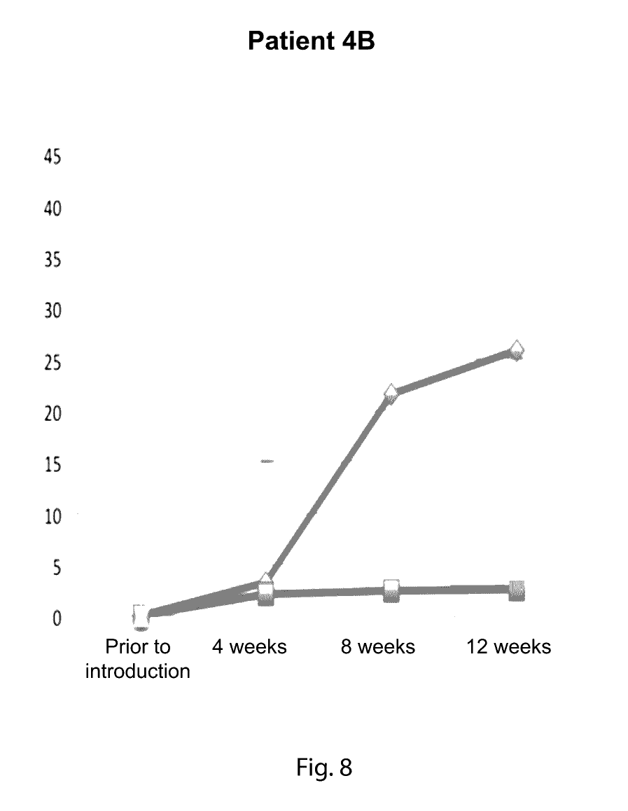

Relative reduction of wrinkles of Patient 4B after the introduction of the modified autogenous fibroblasts is shown in FIG. 8.

The clinical aging index CAI after 12 weeks was 1.928+0.631.

Table 9 contains the data of assessing the facial skin parameters obtained in the course of questioning and examining the patients.

TABLE 9

| | | No. | | | |
|---|---|---|---|---|---|
| | | 1B | 2B | 3B | 4B |
| | | Age | | | |
| | | 48 | 55 | 68 | 61 |
| | | Sex | | | |
| | | Female | Female | Female | Male |
| Texture | 4 weeks | 0 | 0 | 0 | 0 |
| | 8 weeks | 0 | 0 | − | − |
| | 12 weeks | 0 | − | + | ++ |
| Moisture | 4 weeks | − | 0 | + | + |
| | 8 weeks | + | 0 | 0 | 0 |
| | 12 weeks | + | + | 0 | 0 |
| Elasticity | 4 weeks | 0 | 0 | 0 | 0 |
| | 8 weeks | + | + | + | 0 |
| | 12 weeks | + | ++ | + | + |
| Swelling | 4 weeks | 0 | 0 | 0 | 0 |
| | 8 weeks | + | + | + | + |
| | 12 weeks | ++ | + | + | ++ |
| Vascular pattern | 4 weeks | 0 | 0 | 0 | 0 |
| | 8 weeks | + | + | + | 0 |
| | 12 weeks | + | + | + | ++ |

Signs in the Table:
“+” stands for improvement
“−” stands for deterioration
“++” stands for significant improvement
“−−” stands for significant deterioration
“0” stands for no dynamics

All the four patients of Group B evaluate the evidence of changes positively.

The objective data of examination of all the patients proved as follows: improved skin tightness and texture, reduced and smoothed facial pores, significantly reduced couperosis.

The decrease of clinical signs of facial skin aging was accompanied by the improvement of functional skin parameters. Thus, after 12 weeks we observed the following changes:

Patient 1B with the tired skin type ticked 11 clinical aging signs of the questionnaire the following boxes: disappeared swelling after sleep, as well as smoothed nasolabial fold and disappeared periorbital reticular wrinkles.

Patient 2B with the small lines aging type ticked 11 clinical aging signs of the questionnaire the following boxes: visibly levelled facial contours, smoothed periorbital wrinkles, levelled glabellar muscle folds, levelled upper lip border, significantly increased skin tightness by week 8, smoothed contours of the lower third of the face.

Patient 3B with the deforming aging type ticked 11 clinical aging signs of the questionnaire the following boxes: improved skin texture, small capillary network on the cheeks disappeared by 80%. In the lower third of the periorbital area, she observed smoothed hernial sacs of the lower eyelids, disappeared morning eyelid swelling, smoothed wrinkles in the periorbital area (crow's feet), smoothed wrinkles of the lower mouth corners. At the same time, she did not observe any changes in terms of the skin tightness and moisture in general.

Patient 4B with the deforming aging type ticked 11 clinical aging signs of the questionnaire the following boxes: significantly reduced facial swelling by week 12 of the follow-up supervision, significantly improved skin texture, disappeared vascular pattern on the nose wings and the left cheek, smoothed nasolabial fold, smoothed wrinkles under the eyes.

After comparing the experimental findings relating to the patients of Group A (see Table 4) and Group B (see Table 9) we may conclude as follows.

When comparing dynamic values of the patients of Group A and Group B by conducting the functional diagnostic profilometry, we observed that the patients of Group B had significant improvement of the “relative reduction of wrinkles” sign versus the patients of Group A. During the follow-up visit, the patients of Group B on week 8 and week 12 had evident morphological signs of the increased skin elasticity, which was also proved by the profilometric values, as well as the assessment data based on the questioning of the patients of Group B.

All the said changes may be evaluated as the evidence of improvement in terms of the signs of aging for the patients of Group B, i. e. those to whom the modified fibroblasts were injected.

The claimed method does not imply the embedding of the exogenous genetic material in the cell genome.

Therefore, the mentioned embodiments confirm the accomplishment of the set objective, i. e. the creation of the method for correcting pathological human skin conditions caused by aging, which takes account of all genetic defects, as well as damaged gene expressions, if any, or mutations in the gene DNA structure changing the activity or function of the proteins coded by these genes. At the same time, the solution aims at not simply replenishing the quantity of cells, but also at compensating for their defects, or the mutations in the gene structure changing the protein activity or function.

These embodiments also prove the clinical applicability of the claimed invention.

What is claimed is:

1. A method for correcting pathological human skin conditions caused by aging comprising the using of a patient's autogenous fibroblasts and introducing them to the patient, the method comprising:
assessing the patient's skin, sampling a cell material and growing cells to isolate patient's fibroblast culture;
separating the cell material into a first part and a second part, sending the first part of the fibroblast culture for analysis and storing the second part;
analysing the fibroblast culture by determining a DNA sequence and activity of the genes selected from the group consisting of GFB1, TGFBR2, COL1A1, COL1A2, SOD1, SOD2, GPX1, GPX3, CLCA2;
comparing findings of the analysing with normal DNA sequences and normal expression level data of the respective genes to determine deviations in the fibroblast genome;
utilizing the findings to determine a connection of the deviations in a gene structure and/or function with changes of the patient's skin;
compensating for the deviations by creating genetic constructs with cDNA of the patient's genes the activity of which is changed, or the DNA of which has deviations to ensure that the structure and functions of said genes are as those of normal ones; and
embedding the genetic constructs in the patient's fibroblast culture and injecting modified autogenous fibroblasts into the patient.

2. The method of claim 1, further comprising modifying the patient's fibroblast culture with the vectors containing the cDNA of the patient's genes the activity of which is changed, or the DNA of which has deviations to place the respective cDNAs in the created genetic constructs that are used for transfection into the patient's cells.

3. The method of claim 2, wherein the genetic construct is used in combination with dendrimeric macromolecules.

4. The method of claim 2, wherein the genetic construct is used in combination with liposomes.

5. The method of claim 2, wherein the genetic construct is used in combination with amphiphilic block copolymers.

6. The method of claim 1, wherein the skin is assessed using functional diagnostics of measuring instruments allowing to obtain quantitative parameters characterizing the patient's skin condition.

7. The method of claim 1, wherein sampling the material occurs in a UV-protected area.

8. The method of claim 1, wherein during the step of comparing the findings of the analysing with normal DNA sequences and normal level data of the respective genes to determine deviations in the fibroblast expression genome, gene sequences from a GenBank database and gene expression data from an UniGene database are accepted as normal DNA sequences and the data of the normal expression level of the respective genes.
